(19) United States
(12) Patent Application Publication
(10) Pub. No.: US 2009/0208509 A1
(43) Pub. Date: Aug. 20, 2009

(54) Pharmaceutical formulation of an antibody against IL-1R

(75) Inventors: Hanns-Christian Mahler, Basel (CH); Robert Mueller, Basel (CH); Christine Wurth, Loerrach (CH)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 12/284,098

(22) Filed: Sep. 18, 2008

(30) Foreign Application Priority Data
Sep. 21, 2007 (EP) 07116980.0

(51) Int. Cl. A61K 39/395

(52) U.S. Cl. 424/142.1; 424/158.1

(57) ABSTRACT

Pharmaceutical formulation of an antibody against IL-1R (interleukin-1 receptor), a process for the preparation and uses of the formulation.

### CROSS REFERENCE TO PRIOR APPLICATIONS

This application claims the benefit of priority under 35 USC §119 to European Application No. EP 07116980.0 filed on Sep. 21, 2007, the contents of which are hereby incorporated in their entirety by reference.

### FIELD OF THE INVENTION

The present invention relates to a pharmaceutical formulation of an antibody against IL-1R (interleukin-1 receptor), a process for the preparation and uses of the formulation.

### BACKGROUND OF THE INVENTION

The interleukin-1 receptor (IL-1R) is involved with transmission of inflammatory effects associated with interleukin-1 (IL-1) ligands. Modulation of IL-1R can thus provide a mechanism for treatment of inflammatory conditions such as rheumatoid arthritis, osteoarthritis, and the like. Antibodies against IL-1R are known from, e.g., U.S. Pat. No. 6,511,665. Pharmaceutical formulations of antibodies against IL-1R would be useful for treatment of inflammatory conditions.

### SUMMARY OF THE INVENTION

In a first aspect, the invention relates to a pharmaceutical formulation comprising:

1 to 150 mg/mL of an antibody against IL-1R;

1 to 100 mM of a buffer;

optionally 0.001 to 1% of a surfactant; and

(a) 10 to 500 mM of a stabilizer; or

(b) 10 to 500 mM of a stabilizer and 1 to 800 mM of a tonicity agent; or

(c) 1 to 800 mM of a tonicity agent;

at a pH in the range of from 5.0 to 7.0,

The formulation according to the invention can be in a liquid form, a lyophilized form or in a liquid form reconstituted from a lyophilized form.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The following definitions are set forth to illustrate and define the meaning and scope of the various terms used to describe the invention herein.

The term “buffer” as used herein denotes a pharmaceutically acceptable excipient, which stabilizes the pH of a pharmaceutical preparation. Suitable buffers are known in the art and can be found in the literature. Preferred pharmaceutically acceptable buffers comprise but are not limited to histidine-buffers, other amino acid-buffers, citrate-buffers, succinate-buffers, acetate-buffers and phosphate-buffers or mixtures thereof. Still preferred buffers comprise L-histidine or mixtures of L-histidine and L-histidine hydrochloride with pH adjustment with an acid or a base known in the art. The abovementioned buffers are generally used in an amount of about 1 mM to about 100 mM, preferably of about 5 mM to about 50 mM and more preferably of about 10-20 mM. Independently from the buffer used, the pH can be adjusted at a value comprising about 5.0 to about 7.0 and preferably about 5.5 to about 6.5 and still preferably about 6.0 with an acid or a base known in the art, e.g. hydrochloric acid, acetic acid, phosphoric acid, sulfuric acid and citric acid, sodium hydroxide and potassium hydroxide or the pH can be achieved by mixing acid and salt or base and salt in predefined ratios.

The term “surfactant” as used herein denotes a pharmaceutically acceptable excipient which is used to protect protein formulations against interfacial stresses like agitation and shearing. Examples of pharmaceutically acceptable surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulphate (SDS). Preferred polyoxyethylenesorbitan-fatty acid esters are polysorbate 20, (sold under the trademark Tween 20) and polysorbate 80 (sold under the trademark Tween 80™). Preferred polyethylene-polypropylene copolymers are those sold under the names Pluronic® F68 or Poloxamer 188™. Preferred Polyoxyethylene alkyl ethers are those sold under the trademark Brij™. Preferred alkylphenolpolyoxyethylene esthers are sold under the tradename Triton-X. When polysorbate 20 (Tween 20™) and polysorbate 80 (Tween 80™) are used they are generally used in a concentration range of about 0.001 to about 1%, preferably of about 0.005 to about 0.1% and more preferably about 0.01% to about 0.04% w/v (weight/volume).

The term “stabilizer” denotes a pharmaceutical acceptable excipient, which protects the active pharmaceutical ingredient and/or the formulation from chemical and/or physical degradation during manufacturing, storage and application. Chemical and physical degradation pathways of protein pharmaceuticals are reviewed by Cleland et al. (1993), Crit. Rev Ther Drug Carrier Syst 10(4):307-77, Wang (1999) Int J Pharm 185(2): 129-88, Wang (2000) Int J Pharm 203(1-2):1-60 and Chi et al. (2003) Pharm Res 20(9):1325-36. Stabilizers include but are not limited to sugars, amino acids, polyols, cyclodextrines, e.g. hydroxypropyl-p-cyclodextrine, sulfobutylethyl-β-cyclodextrin, β-cyclodextrin, polyethylenglycols, e.g. PEG 3000, PEG 3350, PEG 4000, PEG 6000, albumine, human serum albumin (HSA), bovine serum albumin (BSA), salts, e.g. sodium chloride, magnesium chloride, calcium chloride, chelators, e.g. EDTA as hereafter defined. As mentioned hereinabove, stabilizers can be present in the formulation in an amount of about 10 to about 500 mM, preferably in an amount of about 10 to about 300 mM and more preferably in an amount of about 100 mM to about 300 mM.

A subgroup within the “stabilizers” are lyoprotectants. The term “lyoprotectant” denotes pharmaceutical acceptable excipients, which protect the labile active ingredient (e.g. a protein) against destabilizing conditions during the lyophilisation process, subsequent storage and reconstitution. Lyoprotectants comprise but are not limited to the group consisting of sugars, polyols (such as e.g. sugar alcohols) and amino acids. Preferred lyoprotectants can be selected from the group consisting of sugars such as sucrose, trehalose, lactose, glucose, mannose, maltose, galactose, fructose, sorbose, raffinose, neuraminic acid, amino sugars such as glucosamine, galactosamine, N-methylglucosamine (“Meglumine”), polyols such as mannitol and sorbitol, and amino acids such as arginine and glycine. Lyoprotectants are generally used in an amount of about 10 to 500 mM, preferably in an amount of about 10 to about 300 mM and more preferably in an amount of about 100 to about 300 mM.

Another subgroup within the “stabilizers” are antioxidants. The term “antioxidant” denotes pharmaceutically acceptable excipients, which prevent oxidation of the active pharmaceutical ingredient. Antioxidants comprise but are not limited to ascorbic acid, glutathione, cysteine, methionine, citric acid, EDTA. Antioxidants can be used in an amount of about 1 to about 100 mM, preferably in an amount of about 5 to about 50 mM and more preferably in an amount of about 5 to about 20 mM.

The term “sugar” as used herein denotes a monosaccharide or an oligosaccharide. A monosaccharide is a monomeric carbohydrate which is not hydrolysable by acids, including simple sugars and their derivatives, e.g. aminosugars. Examples of monosaccharides include glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a chain. The monomeric saccharide units within an oligosaccharide can be identical or different. Depending on the number of monomeric saccharide units the oligosaccharide is a di-, tri-, tetra-penta- and so forth saccharide. In contrast to polysaccharides the monosaccharides and oligosaccharides are water soluble. Examples of oligosaccharides include sucrose, trehalose, lactose, maltose and raffinose. Preferred sugars are sucrose and trehalose, most preferred is trehalose.

The term “amino acid” as used herein denotes a pharmaceutically acceptable organic molecule possessing an amino moiety located at α-position to a carboxylic group. Examples of amino acids include arginine, glycine, ornithine, lysine, histidine, glutamic acid, asparagic acid, isoleucine, leucine, alanine, phenylalanine, tyrosine, tryptophane, methionine, serine, proline. Amino acids are generally used in an amount of about 10 to 500 mM, preferably in an amount of about 10 to about 300 mM and more preferably in an amount of about 100 to about 300 mM.

The term “polyols” as used herein denotes pharmaceutically acceptable alcohols with more than one hydroxy group. Suitable polyols comprise to but are not limited to mannitol, sorbitol, glycerine, dextran, glycerol, arabitol, propylene glycol, polyethylene glycol, and combinations thereof. Polyols can be used in an amount of about 10 mM to about 500 mM, preferably in an amount of about 10 to about 300 mM and more preferably in an amount of about 100 to about 300 mM.

The term “tonicity agents” as used herein denotes pharmaceutically acceptable tonicity agents. Tonicity agents are used to modulate the tonicity of the formulation. The formulation can be hypotonic, isotonic or hypertonic. Isotonicity in general relates to the osmostic pressure relative of a solution usually relative to that of human blood serum. The formulation according to the invention can be hypotonic, isotonic or hypertonic but will preferably be isotonic. An isotonic formulation is liquid or liquid reconstituted from a solid form, e.g. from a lyophilised form and denotes a solution having the same tonicity as some other solution with which it is compared, such as physiologic salt solution and the blood serum. Suitable tonicity agents comprise but are not limited to sodium chloride, potassium chloride, glycerine and any component from the group of amino acids, sugars, in particular glucose. Tonicity agents are generally used in an amount of about 1 mM to about 800 mM.

Within the “stabilizers” and “tonicity agents” there is a group of compounds which can function in both ways, i.e. they can at the same time be a stabilizer and a tonicity agent. Examples thereof can be found in the group of sugars, amino acids, polyols, cyclodextrines, polyethylenglycols and salts. An example for a sugar which can at the same time be a stabilizer and a tonicity agent is trehalose.

The term “adjuvants” as used herein denotes preservatives, wetting agents, emulsifying agents and dispersing agents. Prevention of presence of microorganisms may be ensured both by sterilization procedures, and by the inclusion of various antibacterial and antifingal agents, for example, paraben, chlorobutanol, phenol, sorbic acid, and the like. Preservatives are generally used in an amount of about 0.001 to about 2% (w/v). Preservatives comprise but are not limited to ethanol, benzyl alcohol, phenol, m-cresol, p-chlor-m-cresol, methyl or propyl parabens, benzalkonium chloride.

The term “liquid” as used herein in connection with the formulation according to the invention denotes a formulation which is liquid at a temperature of at least about 2 to about 8° C. under atmospheric pressure.

The term “lyophilizate” as used herein in connection with the formulation according to the invention denotes a formulation which is manufactured by freeze-drying methods known in the art per se. The solvent (e.g. water) is removed by freezing following sublimation under vacuum and desorption of residual water at elevated temperature. The lyophilisate has usually a residual moisture of about 0.1 to 5% (w/w) and is present as a powder or a physical stable cake. The lyophilizate is characterized by a fast dissolution after addition of a reconstitution medium.

The term “reconstituted formulation” as used herein in connection with the formulation according to the invention denotes a formulation which is lyophilized and re-dissolved by addition of reconstitution medium. The reconstitution medium comprise but is not limited to water for injection (WFI), bacteriostatic water for injection (BWFI), sodium chloride solutions (e.g. 0.9% (w/v) NaCl), glucose solutions (e.g. 5% glucose), surfactant, containing solutions (e.g. 0.01% polysorbate 20), a pH-buffered solution (eg. phosphate-buffered solutions).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

### Antibodies

Exemplary antibodies against IL-1R usable in the formulations of the invention are described in WO2005/023872 and include antibodies which are characterized in comprising as heavy chain complementarity determining regions (CDRs) the CDRs of SEQ ID NO:1 and as light chain CDRs the CDRs of SEQ ID NO:2.

The CDR sequences can be determined according to the standard definition of Kabat et al., Sequences of Proteins of Immunological Interest, 5th ed., Public Health Service, National Institutes of Health, Bethesda, Md. (1991). On this basis, the CDRs have the following sequences:

Heavy chain CDRs:

CDR1 (aa 45-54) of SEQ ID NO:1,

CDR2 (aa 69-84) of SEQ ID NO: 1,

CDR3 (aa 117-123) of SEQ ID NO: 1,

Light chain CDRs:

CDR1 (aa 43-57) of SEQ ID NO:2,

CDR2 (aa 73-79) of SEQ ID NO:2, and

CDR3 (aa 112-120) of SEQ ID NO:2.

The antibody may be obtained from hybridoma cell line MAK<h-IL-1RI>2D8 (DSM ACC 2601) or is a chimeric, humanized or T cell epitope depleted antibody variant or a fragment of said antibody, showing an IC-50 value of 35 pM or lower for the inhibition of IL-1 mediated secretion of 11-8 in human fibroblast cells MRC5 (ATCC CCL 171).

Antibodies useful in the invention may be of the IgG₄ isotype, e.g. carrying a mutation of serine 228 to proline (Angal et al., Mol. Immunol. 30 (1993) 105-108). Alternatively said antibodies may be of IgG₁ isotype and may be modified in the hinge region at about aa 220-240 between CH1 and CH2 and/or the second inter-domain region of about aa 330 between CH2 and CH3 (numbering according to Kabat, see e.g. Johnson and Wu, Nucleic Acids Res. 28 (2000) 214-218) to avoid effector function. Switching of IgG class can be easily performed by exchange of the constant heavy and light chains of the antibody by heavy and light chains from an antibody of the desired class, like IgG₁ or IgG₄. Such methods are known in the state of the art.

In one embodiment the antibody is of rat origin and comprises the antibody sequence frame of a rat antibody according to Kabat. Preferably in the Kabat sequences amino acid 10 (serine) is deleted from the VL chain (DEL10) and/or amino acid 26 (glycine) of the VH chain is changed to glutamic acid (G26E). Preferably the antibody is T cell epitope depleted using methods described in WO 98/08097.

The constant region in one embodiment is a human IgG₁ or human IgG₄ constant region according to Kabat. Preferred constant regions are shown in SEQ ID NO:29, 30 and 31.
SEQ ID NO:1:variable region of heavy chain of rat 2D8; aa1-19 signal sequence, 20-134 variable region, 135-139 terminal fragment of rat origin
SEQ ID NO:2:variable region of light chain of rat 2D8; aa 1-20 signal sequence, 21-129 variable region, 130-138 terminal fragment of rat origin
SEQ ID NO:3:amino acid sequence of 2D8 chimeric H-chain (rat/human) (IgG1)
SEQ ID NO:4: amino acid sequence of 2D8 chimeric H-chain (rat/human) (IgG4)
SEQ ID NO:5:amino acid sequence of 2D8 chimeric L-chain (rat/human)

The antibody is preferably a monoclonal antibody and, in addition, a chimeric antibody (human constant chain), a humanized antibody and especially preferably a T cell epitope depleted antibody.

The antibody binds to IL-1R human in competition to the antibodies characterized by the variable chains of SEQ ID NO:1 or 2.

The antibody may be further characterized by an affinity of 300 pM or less, preferably 200 pM (K_{D}) or less and more preferably of about 70-200 pM.

The hybridoma cell line MAK<h-IL-1RI>2D8 was deposited with Deutsche Sammlung von Mikroorganismen und Zellkulturen GmbH (DSMZ), Germany, on Jul. 10, 2003 under Accession No. DSM ACC 2601.

An antibody obtainable from said cell line is one preferred antibody in the composition of the invention. Preferred are also all antibodies which can be combined from the variable and constant regions shown in SEQ ID NO:6 to 12, 13 to 19, 20 to 23, 24 to 27 and SEQ ID NO:29, 30 and 31 and showing an IC-50 value of 35 pM or lower for the inhibition of IL-1 mediated secretion of I1-8 in human fibroblast cells MRC5 (ATCC CCL 171). These sequences are examples of sequences which were obtained by modifying the sequence of antibody 2D8 in order to get improved antibodies retaining the superior properties of antibody 2D8 which are IC₅₀ and/or epitope characteristics. In such antibodies, light chains and heavy chains from SEQ ID NO:6 to 12, 13 to 19, (T cell epitope depleted) or from SEQ ID NO: 20 to 23, 24 to 27 (humanized) are combined with constant region from SEQ ID NO:29 and SEQ ID NO:30 or 31. Especially preferred are antibodies DEI5/7, DEI4/7, DEI2/4, DEI5/4, DEI4/5, DEI5/5, HUM2/2, HUM2/3, DEI1/8, DEI2/8, DEI2/9, DEI4/9, DEI5/8 and DEI5/9.

The antibodies useful in the formulations according to the invention may be produced by recombinant means, e.g. by those described in WO2005/023872. Such methods are widely known in the state of the art and comprise protein expression in prokaryotic and eukaryotic cells with subsequent isolation of the antibody polypeptide and usually purification to a pharmaceutically acceptable purity. For the protein expression, nucleic acids encoding light and heavy chains or fragments thereof are inserted into expression vectors by standard methods. Expression is performed in appropriate prokaryotic or eukaryotic host cells like CHO cells, NS0 cells, SP2/0 cells, HEK293 cells, COS cells, yeast, or E. coli cells, and the antibody is recovered from the cells (supernatant or cells after lysis) by standard techniques, including alkaline/SDS treatment, CsCl banding, column chromatography, agarose gel electrophoresis, and others known in the art, e.g. as described in WO2005/023872.

### Formulations

The invention relates to a pharmaceutical formulation comprising:

1 to 150 mg/mL of an antibody against IL-1R;

1 to 100 mM of a buffer;

optionally 0.001 to 1% of a surfactant; and

(a) 10 to 500 mM of a stabilizer; or

(b) 10 to 500 mM of a stabilizer and 1 to 800 mM of a tonicity agent; or

(c) 1 to 800 mM of a tonicity agent;

at a pH in the range of from 5.0 to 7.0.

In one embodiment the invention provides a formulation containing:

1 to 150 mg/mL human monoclonal antibody (huMab) IL-1R,

0.01% to 0.04% Tween 20 w/v, 15 mM L-histidine, and

90 mM to 160 mM trehalose, at pH 5.8±0.5.

In another embodiment the invention provides a formulation containing:

25 mg/mL huMab IL-1R,

0.01% polysorbate 20,

15 mM L-histidine, and

90 mM trehalose, at pH 5.8±0.5.

In another embodiment the invention provides a formulation containing:

25 mg/mL huMab IL-1R,

0.04% polysorbate 20,

15 mM L-histidine, and

90 mM trehalose, at pH 5.8±0.5.

In another embodiment the invention provides a formulation containing:

25 mg/mL huMab IL-1R,

0.01% polysorbate 20,

15 mM L-histidine, and

160 mM trehalose, at pH 5.8±0.5.

In another embodiment the invention provides a formulation containing:

25 mg/mL huMab IL-1R,

0.04% polysorbate 20,

15 mM L-histidine, and

160 mM trehalose, at pH 5.8±0.5.

In another embodiment the invention provides a formulation containing:

25 mg/mL huMab IL-1R,

0.025% polysorbate 20,

15 mM L-histidine, and

125 mM trehalose, at pH 5.8±0.5.

In another embodiment the invention provides a formulation containing:

25 mg/mL huMab IL-1R,

0.01% polysorbate 20,

15 mM L-histidine, and

96 mM trehalose, at pH 5.8±0.5.

In another embodiment the invention provides a formulation containing:

25 mg/mL huMab IL-1R,

0.01% polysorbate 20,

15 mM L-histidine, and

125 mM trehalose, at pH 5.8±0.5.

In another embodiment the invention provides a formulation containing:

25 mg/mL huMab IL-1R,

0.025% polysorbate 20,

15 mM L-histidine, and

90 mM trehalose, at pH 5.8±0.5.

In another embodiment the invention provides a formulation containing:

25 mg/mL huMab IL-1R,

0.025% polysorbate 20,

15 mM L-histidine, and

160 mM trehalose, at pH 5.8±0.5.

The formulation according to the invention preferably comprises about 0.001 to about 1% of at least one surfactant.

The formulation according to the invention can be administered by intravenous (i.v.), subcutaneous (s.c.), intra-articular (i.a.) or any other parental administration means such as those known in the pharmaceutical art.

In one embodiment the formulation comprises one or more isotonicity agents in an amount of about 5 mM to about 800 mM.

In one embodiment the formulation comprises a sugar in an amount of about 25 mM to about 500 mM.

The formulation according to the invention can be in a liquid form, a lyophilized form or in a liquid form reconstituted from a lyophilized form.

In one embodiment the present invention provides a formulation wherein the antibody is present in an amount in the range of from 10 to 150 mg/mL, preferably from 10 to 50 mg/mL.

Lyophilized formulations can be reconstituted in less volume to achieve IL-1R concentrations of >25 mg/mL, eg. 50 mg/mL.

The composition must be sterile and fluid to the extent that the composition is deliverable by syringe. In addition to water, the carrier can be an isotonic buffered saline solution, ethanol, polyol (e.g., glycerol, propylene glycol, and liquid polyetheylene glycol, and the like), and suitable mixtures thereof.

The formulation according to the invention can be administered by intravenous (i.v.), subcutaneous (s.c.) or any other parental administration means such as those known in the pharmaceutical art.

The formulation according to the invention can be prepared by methods known in the art, e.g. ultrafiltration-diafiltration, dialysis, addition and mixing, lyophilisation, reconstitution, and combinations thereof. Examples of preparations of formulations according to the invention can be found hereinafter.

The formulations according to the invention have new and inventive properties causing a benefit for a patient suffering from rheumatoid arthritis and/or osteoarthritis.

The invention further comprises the use of a formulation according to the invention for the manufacture of a medicament for rheumatoid arthritis and/or osteoarthritis treatment.

The invention further provides methods for treating rheumatoid arthritis and/or osteoarthritis comprising administering to a patient diagnosed as having rheumatoid arthritis (and therefore being in need of such a therapy) an effective amount of a composition of the invention.

In addition, the invention comprises a method for the manufacture of a pharmaceutical composition according to the invention.

In one embodiment, the pharmaceutical composition may be included in an article of manufacture or kit.

A composition of the present invention can be administered by a variety of methods known in the art. As will be appreciated by the skilled artisan, the route and/or mode of administration will vary depending upon the desired results.

To administer a composition of the invention by certain routes of administration, it may be necessary to dilute the composition in a diluent. Pharmaceutically acceptable diluents include saline, glucose, Ringer and aqueous buffer solutions.

### EXAMPLES

Examples of the formulations encompassed by the present invention and within the scope of the invention are provided in the following examples. These examples are provided to enable those skilled in the art to more clearly understand and to practice the present invention. They should not be considered as limiting the scope of the invention, but merely as being illustrative and representative thereof.

### Example 1

### Liquid and Lyophilised Drug Product Formulations

Liquid and lyophilised drug product formulations for subcutaneous or intra-articular administration according to the invention were developed as follows:

Preparation of liquid formulations. Solutions of approx. 10 mg/ml huMab IL-1R in the production buffer (e.g. 20 mM histidine buffer at pH approx. 6.0) were processed by tangential flow filtration (TFF) to increase the protein concentration above target protein concentration. The protein and buffer concentration was adjusted to the desired con-centrations by dilution with buffer and water for injection. Sugars for stabilizing the protein and for tonicity adjustment were added as required. Surfactant was added to the formulations after processing by TFF as 100 to 400-fold stock solutions. All formulations were sterile filtered through 0.2 μm low protein binding filters and aseptically aliquoted into sterile glass vials and closed with Teflon-coated rubber stoppers and alucrimp caps. These formulations were stored at different temperatures for different intervals of time and removed for analysis at the timepoints indicated in the individual paragraphs. Formulations were analyzed 1) by UV spectrophotometry, 2) by Size Exclusion Chromatography (SEC), 3) by Ion exchange chromatography (IEC), 4) for visible and subvisible particles and 5) by turbidity of the solution.

Preparation of lyophilised formulations. Solutions of 25 mg/ml huMab IL-1R were prepared as described above for liquid formulations. Any lyophilisation method known in the art is intended to be within the scope of the invention. For example, the lyophilisation process used for this study included the cooling of the formulation from room temperature to approx 5° C. (pre-cooling) followed by a freezing at −40° C. (Freeze I) at a ramping rate of about 1° C./min to 5° C./min. The first drying step can take place at a ramping rate of 0.3 to 0.5° C./min from −40° C. to −30° C. and then hold at −30° C. for at least 50 hours at a chamber pressure of approx. 75 to 80 mTorr. A second drying step can take place at a ramping rate of 0.1 to 0.3° C./min from −30° C. to 25° C. and hold at 25° C. for at least 5 hours at a chamber pressure of about 50 to 80 mTorr (the applied drying schedule is provided in Table 1 and Table 2). huMab IL-1R formulations which were dried using the described lyophilisation processes were found to have conveniently quick reconstitution times of about 2-4 minutes when reconstituted from 25 mg/mL to a 50 mg/mL protein concentration. Lyophilisation was carried out in an Usifroid SMH90/16039 Freeze-dryer (Usifroid, France). All lyophilised cakes in this study had a residual water content of approximately 0.1 to 1.0% as determined by Karl-Fischer method. The lyophilised vials were stored at different temperatures for different intervals of time. The lyophilised formulations were reconstituted with the respective volume of water for injection (WFI) prior to 1) analysis by UV spectro-photometry, 2) determination of the reconstitution time, 3) analysis by Size Exclusion Chromatography (SEC) 4) by Ion exchange chromatography (IEC), 5) determination of subvisible and visible particles and 6) by turbidity of the solution.

SEC was performed to detect soluble high molecular weight species (aggregates) and low molecular weight hydrolysis products in the formulations. The method used a Merck Hitachi 7000 HPLC instrument for the liquid samples and the lyophilized samples. The instrument was equipped with a TSK G3000 SWXL column; the method used 0.2M K₂HPO₄/0.25M KCL, pH 7.0 as mobile phase.

Ion Exchange Chromatography (IEC) was performed to detect chemical degradation products altering the net charge of IL-1R in the formulations. The method used a Waters Alliance 2795 with UV detector for the liquid samples and the lyophilized samples. The instrument was equipped with a Dionex ProPac WCX-10, 4 mm×250 mm and 10 mM Sodium Phosphate buffer pH 6.0 and 10 mM Sodium Phosphate buffer pH 6.0+0.75M NaCl as mobile phases.

The UV spectroscopy for determination of the protein concentration was performed on a Varian Cary Bio UV spectrophotometer at 280 nm for both the liquid samples and lyophilized samples.

For the determination of the turbidity, opalescence was measured in FTU (turbidity units) using a HACH 2100AN turbidimeter at room temperature.

Samples were analyzed for subvisible particles by using a H/AC Royco PharmaSpec (HRLD-150), and for visible particles by using a Seidenader V90-T visual inspection instrument.

Table 1 below freeze-drying cycle information used with the formulations of this example. Tables 2-13 below provide details for several formulations prepared using the procedure of this example.

TABLE 1
Freeze-drying Cycle

| | | | | Vacuum Set |
|---|---|---|---|---|
| | Shelf temperature | Ramp Rate | Hold time | point |
| Step | (° C.) | (° C./min) | (min) | (mTorr) |
| Pre-cooling | 5° C. | 0.0 | 60 | — |
| Freeze I | −40° C. | 1.0 | 120 | — |
| Prim Drying | −30° C. | 0.5 | 3720 | 80 |
| Sec Drying | +25° C. | 0.2 | 300 | 80 |

TABLE 2
Formulation of 25 mg/mL MAB IL-1R, 15 mM His/HCl,
160 mM Trehalose, 0.01% w/v Polysorbate 20, pH 5.8

| | Storage | | SEC | | | IEC - |
|---|---|---|---|---|---|---|
| Storage | Time | Protein | (Mono- | | Turbid- | significant |
| cond. | (months) | (mg/mL) | mer %) | pH | ity | changes |
| Initial | | 25.3 | 99.0 | 5.9 | 7.5 | No |
| 2-8° C. | 3 | 24.1 | 99.0 | 5.8 | 7.0 | No |
| | 6 | 23.7 | 98.9 | 5.8 | 7.2 | No |
| | 12 | 24.8 | 98.9 | 5.8 | 6.5 | no |
| 25° C. | 3 | 23.7 | 98.9 | 5.8 | 7.5 | No |
| | 6 | 23.9 | 98.7 | n/d | 7.8 | Small changes |
| | 12 | 24.8 | 98.4 | 5.8 | 6.6 | Yes |
| 40° C. | 3 | n/a | 96.2 | n/d | n/d | Yes |
| −20° C. | 3 | 24.1 | 99.0 | 5.8 | 6.8 | No |
| | 6 | 23.4 | 99.0 | n/d | 7.2 | No |
| | 12 | 24.9 | 99.0 | 5.8 | 6.6 | No |
| −80° C. | 3 | 23.4 | 99.0 | 5.8 | 7.2 | No |
| | 6 | 23.9 | 99.0 | 5.8 | 6.9 | No |
| Shaking | 1 week | 24.3 | 98.95 | 5.8 | 7.1 | n/d |
| 25° C. | | | | | | |

n/d = not determined

TABLE 3
Formulation of 25 mg/mL MAB IL-1R, 15 mM His/HCl,
90 mM Trehalose, 0.01% w/v Polysorbate 20, pH 5.8

| | Storage | | SEC | | | IEC - |
|---|---|---|---|---|---|---|
| Storage | Time | Protein | (Mono- | | Turbid- | significant |
| cond. | (months) | (mg/mL) | mer %) | pH | ity | changes |
| Initial | 0 | 24.7 | 99.0 | 5.9 | 7.4 | No |
| −20° C. | 12 | 25.0 | 99.0 | n/d | 6.9 | No |
| 2-8° C. | 12 | 24.8 | 98.9 | 5.8 | 7.5 | No |
| 25° C. | 12 | 24.5 | 98.3 | 5.8 | 7.2 | Yes |
| 40° C. | 3 | n/d | 96.1 | n/d | n/d | Yes |
| Shaking | 1 week | 23.7 | 99.0 | 5.8 | 7.1 | n/d |
| 25° C. | | | | | | |

n/d = not determined

TABLE 4
Formulation of 25 mg/mL MAB IL-1R, 15 mM His/HCl,
90 mM Trehalose, 0.04% w/v Polysorbate 20, pH 5.8

| | Storage | | SEC | | | IEC - |
|---|---|---|---|---|---|---|
| Storage | Time | Protein | (Mono- | | Turbid- | significant |
| cond. | (months) | (mg/mL) | mer %) | pH | ity | changes |
| Initial | 0 | 24.9 | 99.0 | 5.8 | 7.6 | No |
| −20° C. | 12 | 25.1 | 99.0 | n/d | 7.0 | No |
| 2-8° C. | 12 | 25.0 | 98.9 | 5.8 | 6.8 | No |
| 25° C. | 12 | 25.0 | 97.6 | 5.8 | 9.3 | Yes |
| 40° C. | 3 | n/d | 93.9 | n/d | n/d | Yes |
| Shaking | 1 week | 24.2 | 99.0 | 5.8 | 7.2 | n/d |
| 25° C. | | | | | | |

n/d = not determined

TABLE 5
Formulation of 25 mg/mL MAB IL-1R, 15 mM His/HCl,
160 mM Trehalose, 0.04% w/v Polysorbate 20, pH 5.8

| | Storage | | SEC | | | IEC - |
|---|---|---|---|---|---|---|
| Storage | Time | Protein | (Mono- | | Turbid- | significant |
| cond. | (months) | (mg/mL) | mer %) | pH | ity | changes |
| Initial | 0 | 24.7 | 99.0 | 5.9 | 7.3 | No |
| −20° C. | 12 | 25.0 | 99.0 | n/d | 6.6 | No |
| 2-8° C. | 12 | 25.0 | 99.0 | 5.8 | 6.6 | No |
| 25° C. | 12 | 24.7 | 97.7 | 5.8 | 7.6 | Yes |
| 40° C. | 3 | n/d | 94.3 | n/d | n/d | Yes |
| Shaking | 1 week | 24.6 | 99.0 | 5.8 | 7.0 | n/d |
| 25° C. | | | | | | |

n/d = not determined

TABLE 6
Formulation of 25 mg/mL MAB IL-1R, 15 mM His/HCl, 125
mM Trehalose, 0.025% w/v Polysorbate 20, pH 5.8

| | Storage | | SEC | | | IEC - |
|---|---|---|---|---|---|---|
| Storage | Time | Protein | (Mono- | | Turbid- | significant |
| cond. | (months) | (mg/mL) | mer %) | pH | ity | changes |
| Initial | 0 | 24.3 | 99.0 | 5.9 | 7.7 | No |
| −20° C. | 12 | 25.2 | 99.0 | n/d | 6.5 | No |
| 2-8° C. | 12 | 25.2 | 98.9 | 5.8 | 7.1 | No |
| 25° C. | 12 | 25.3 | 98.2 | 5.8 | 7.1 | Yes |
| 40° C. | 3 | n/d | 95.3 | n/d | n/d | Yes |
| Shaking | 1 week | 24.5 | 99.0 | 5.8 | 7.1 | n/d |
| 25° C. | | | | | | |

n/d = not determined

TABLE 7
Formulation of 25 mg/mL MAB IL-1R, 15 mM His/HCl,
96 mM Trehalose, 0.01% w/v Polysorbate 20, pH 5.8

| | Storage | | SEC | | | IEC - |
|---|---|---|---|---|---|---|
| Storage | Time | Protein | (Mono- | | Turbid- | significant |
| cond. | (months) | (mg/mL) | mer %) | pH | ity | changes |
| Initial | 0 | 24.2 | 99.0 | 5.9 | 8.0 | No |
| −20° C. | 12 | 24.9 | 99.0 | n/d | 7.3 | No |
| 2-8° C. | 12 | 24.9 | 98.9 | 5.8 | 6.8 | No |
| 25° C. | 12 | 24.5 | 98.2 | 5.8 | 7.7 | Yes |
| 40° C. | 3 | n/d | 95.8 | n/d | n/d | Yes |
| Shaking | 1 week | 24.0 | 99.0 | 5.8 | 7.7 | n/d |
| 25° C. | | | | | | |

n/d = not determined

TABLE 8
Formulation of 50 mg/mL MAB IL-1R, 30 mM His/HCl,
320 mM Trehalose, 0.02% w/v Polysorbate 20, pH 5.8

| | | | | | | Recon. | IEC |
|---|---|---|---|---|---|---|---|
| Storage | Storage | Protein | SEC | | | Time | signif. |
| cond. | (months) | (mg/mL) | (Monomer %) | pH | Turbidity | (min) | changes |
| Initial | | 43.1¹ | 99.0 | 5.9 | 10.4 | 2′30 | No |
| 2-8° C. | 1 | 47.7 | 99.0 | 5.9 | 10.6 | 2′00 | No |
| | 3 | 49.8 | 99.0 | 5.8 | 11.1 | 2′50 | No |
| | 6 | 47.8 | 98.9 | 5.8 | 11.2 | 1′40 | No |
| | 12 | 49.6 | 98.9 | 5.8 | 10.9 | 0′40 | No |
| 25° C. | 1 | 48.2 | 98.9 | 5.8 | 11.0 | 2′54 | No |
| | 3 | 48.9 | 98.9 | 5.8 | 10.8 | 5′55 | No |
| | 6 | n/d | 98.8 | n/d | 11.6 | 2′17 | No |
| | 12 | n/d | 98.7 | n/d | 11.4 | 1′00 | No |
| 40° C. | 1 | 48.6 | 98.8 | 5.8 | 11.6 | 1′30 | No |
| | 3 | 50.0 | 98.6 | 5.8 | 11.4 | 2′53 | No |
| | 6 | 47.5 | 98.4 | n/d | 11.1 | 2′45 | No |
| | 12 | 5.04 | 98.1 | n/d | 10.6 | 2′15 | Small changes |

n/d = not determined
¹lyophilizate was reconstituted with 1.2 mL WFI instead of 1.1 mL

TABLE 9
Formulation of 50 mg/mL MAB IL-1R, 30 mM His/HCl,
180 mM Trehalose, 0.02% w/v Polysorbate 20, pH 5.8

| | | | | | | Recon. | IEC |
|---|---|---|---|---|---|---|---|
| Storage | Storage | Protein | SEC | | | Time | signif. |
| cond. | (months) | (mg/mL) | (Monomer %) | pH | Turbidity | (min) | changes |
| Initial | 0 | 45.3¹ | 99.0 | 5.9 | 12.0 | 0′45 | No |
| 2-8° C. | 12 | 51.6 | 98.8 | 5.8 | 12.9 | 2′00 | No |
| 25° C. | 12 | n/a | 98.2 | n/d | 11.5 | 1′40 | No |
| 40° C. | 12 | 51.7 | 96.4 | n/d | 12.9 | 3′55 | Small changes |

n/d = not determined
¹lyophilizate was reconstituted with 1.2 mL WFI instead of 1.1 mL

TABLE 10
Formulation of 50 mg/mL MAB IL-1R, 30 mM His/HCl,
180 mM Trehalose, 0.08% w/v Polysorbate 20, pH 5.8

| | | | | | | Recon. | IEC |
|---|---|---|---|---|---|---|---|
| Storage | Storage | Protein | SEC | | | Time | signif. |
| cond. | (months) | (mg/mL) | (Monomer %) | pH | Turbidity | (min) | changes |
| Initial | 0 | 43.3¹ | 99.0 | 5.9 | 12.3 | 2′15 | No |
| 2-8° C. | 12 | 50.7 | 98.8 | 5.8 | 12.4 | 1′00 | No |
| 25° C. | 12 | n/d | 983 | n/d | 12.6 | 1′15 | No |
| 40° C. | 12 | 50.2 | 96.7 | n/d | 12.9 | 3′45 | Small changes |

n/d = not determined
¹lyophilizate was reconstituted with 1.2 mL WFI instead of 1.1 mL

TABLE 11
Formulation of 50 mg/mL MAB IL-1R, 30 mM His/HCl,
320 mM Trehalose, 0.08% w/v Polysorbate 20, pH 5.8

| | | | | | | Recon. | IEC |
|---|---|---|---|---|---|---|---|
| Storage | Storage | Protein | SEC | | | Time | signif. |
| cond. | (months) | (mg/mL) | (Monomer %) | pH | Turbidity | (min) | changes |
| Initial | 0 | 43.1¹ | 99.0 | 5.9 | 10.1 | 3′46 | No |
| 2-8° C. | 12 | 50.3 | 98.9 | 5.8 | 11.4 | 1′00 | No |
| 25° C. | 12 | n/d | 98.7 | n/d | 11.0 | 2′00 | No |
| 40° C. | 12 | 49.8 | 98.0 | n/d | 9.4 | 1′45 | Small changes |

n/d = not determined
¹lyophilizate was reconstituted with 1.2 mL WFI instead of 1.1 mL

TABLE 12
Formulation of 50 mg/mL MAB IL-1R, 30 mM His/HCl,
250 mM Trehalose, 0.05% w/v Polysorbate 20, pH 5.8

| | | | | | | Recon. | IEC |
|---|---|---|---|---|---|---|---|
| Storage | Storage | Protein | SEC | | | Time | signif. |
| cond. | (months) | (mg/mL) | (Monomer %) | pH | Turbidity | (min) | changes |
| Initial | 0 | 44.2¹ | 99.0 | 5.9 | 10.7 | 3′00 | No |
| 2-8° C. | 12 | 50.4 | 98.9 | 5.8 | 12.0 | 1′00 | No |
| 25° C. | 12 | n/a | 98.6 | n/d | 12.1 | 1′00 | No |
| 40° C. | 12 | 50.2 | 97.7 | n/d | 11.0 | 2′20 | Small changes |

n/d = not determined
¹lyophilizate was reconstituted with 1.2 mL WFI instead of 1.1 mL

TABLE 13
Formulation of 50 mg/mL MAB IL-1R, 30 mM His/HCl,
192 mM Trehalose, 0.02% w/v Polysorbate 20, pH 5.8

| | | | | | | Recon. | IEC |
|---|---|---|---|---|---|---|---|
| Storage | Storage | Protein | SEC | | | Time | signif. |
| cond. | (months) | (mg/mL) | (Monomer %) | pH | Turbidity | (min) | changes |
| Initial | 0 | 46.0¹ | 99.0 | 5.9 | 11.3 | 3′00 | No |
| 2-8° C. | 12 | 51.7 | 98.8 | 5.8 | 11.7 | 0′50 | No |
| 25° C. | 12 | n/d | 98.3 | n/d | 12.4 | 1′45 | No |
| 40° C. | 12 | 50.7 | 96.6 | n/d | 11.9 | 3′25 | Small changes |

n/d = not determined
¹lyophilizate was reconstituted with 1.2 mL WFI instead of 1.1 mL.

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specifications shall be resolved in favor of the latter. Likewise, any conflict between an art-understood definition of a word or phrase and a definition of the word or phrase as specifically taught in this specification shall be resolved in favor of the latter.

We claim:

1. A pharmaceutical formulation comprising:
1 to 150 mg/mL of an antibody against IL-1R;
1 to 100 mM of a buffer;
optionally 0.001 to 1% of a surfactant; and
(a) 10 to 500 mM of a stabilizer; or
(b) 10 to 500 mM of a stabilizer and 1 to 800 mM of a tonicity agent; or
(c) 1 to 800 mM of a tonicity agent;
at a pH in the range of from 5.0 to 7.0,

2. The formulation according to claim 1 wherein the antibody is obtained from a hybridoma cell line.

3. The formulation according to claim 1 which is a liquid formulation.

4. The formulation according to claim 1 which is a lyophilized formulation.

5. The formulation according to claim 1 which is a liquid formulation reconstituted from a lyophilized formulation.

6. The formulation according to claim 1, wherein the antibody concentration is in the range of 10 mg/mL to 150 mg/ml.

7. The formulation according to claim 1, wherein the antibody concentration is in the range of 10 mg/ml to 50 mg/mL.

8. The formulation according to claim 1, wherein the stabilizer is present in the formulation in an amount of 100 mM to 300 mM.

9. The formulation according to claim 1, wherein the stabilizer is trehalose.

10. The formulation according to claim 1, wherein the surfactant is present in the formulation in an amount of 0.005 to 0.1% w/v.

11. The formulation according to claim 1, wherein the surfactant is polysorbate.

12. The formulation according to claim 1, wherein the buffer is present in the formulation in an amount in the range of 5 mM to 50 mM.

13. The formulation according to claim 1, wherein the buffer is a histidine-buffer.

14. The formulation according to claim 1, wherein the pH is 5.5 to 6.0.

15. The formulation according to claim 1, which comprises a tonicity agent.

16. The formulation according to claim 1, wherein the tonicity agent is present in the formulation in an amount in the range of 50 mM to 300 mM.

17. The formulation according to claim 1, wherein the tonicity agent is trehalose.

18. The formulation of claim 1 which comprises:
1 to 150 mg/mL huMab IL-1R,
0.01% to 0.04% Tween 20 w/v,
15 mM L-histidine, and
90 mM to 160 mM trehalose, at pH 5.8±0.5.

19. The formulation of claim 1 which comprises:
25 mg/mL huMab IL-1R, 0.01% polysorbate 20, 15 mM L-histidine, and 90 mM trehalose, at pH 5.8±0.5.

20. The formulation of claim 1 which comprises:
25 mg/mL huMab IL-1R,
0.04% polysorbate 20,
15 mM L-histidine, and
90 mM trehalose, at pH 5.8±0.5.

21. The formulation of claim 1 which comprises:
25 mg/mL huMab IL-1R,
0.01% polysorbate 20,
15 mM L-histidine, and
160 mM trehalose, at pH 5.8±0.5.

22. The formulation of claim 1 which comprises:
25 mg/mL huMab IL-1R,
0.04% polysorbate 20,
15 mM L-histidine, and
160 mM trehalose, at pH 5.8±0.5.

23. The formulation of claim 1 which comprises:
25 mg/mL huMab IL-1R,
0.025% polysorbate 20,
15 mM L-histidine, and
125 mM trehalose, at pH 5.8±0.5.

24. The formulation of claim 1 which comprises:
25 mg/mL huMab IL-1R,
0.01% polysorbate 20,
15 mM L-histidine, and
96 mM trehalose, at pH 5.8±0.5.

25. The formulation of claim 1 which comprises:
25 mg/mL huMab IL-1R,
0.01% polysorbate 20,
15 mM L-histidine, and
125 mM trehalose, at pH 5.8±0.5.

26. The formulation of claim 1 which comprises:
25 mg/mL huMab IL-1R,
0.025% polysorbate 20,
15 mM L-histidine, and 90 mM trehalose, at pH 5.8±0.5.

27. The formulation of claim 1 which comprises:
25 mg/hL huMab IL-1R,
0.025% polysorbate 20,
15 mM L-histidine, and
160 mM trehalose, at pH 5.8±0.5.

28. A method for treating rheumatoid arthritis or osteoarthritis, the method comprising administering to a patient in need thereof an effective amount of a formulation of claim 1.


SEQUENCE LISTING (33 sequences)

SEQ ID NO: 1 - protein, 139 aa, Rattus norvegicus
MAVLGLFFCLLIFPSCVLSQLQLKESGPGLVQPSQTLSLTCTVSELSLTSNSITWIRQPPGRGLEWMGMIWSNGDTDYNSAFTSRLSISRDTSKSQVFLKMNSLQTEDSAMYFCARYNYYFDYWGQGVMVTVSSAETTA

SEQ ID NO: 2 - protein, 138 aa, Rattus norvegicus
METDTLLLWVLLLWVPGSTGDIVLTQAPVLAVSLEQRATISCKASQNVDNRGVSYVHWYQQKPRQQPKLLIYKGSNLAFGVPARFSGSGSRTDFTLTIDPVETDDFATYYCQQSKGHPDTFGAGTKLELKRADAAPTV

SEQ ID NO: 3 - protein, 445 aa, Artificial
  chimeric sequence (rat/human)
QLQLKESGPGLVQPSQTLSLTCTVSELSLTSNSITWIRQPPGRGLEWMGMIWSNGDTDYNSAFTSRLSISRDTSKSQVFLKMNSLQTEDSAMYFCARYNYYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 442 aa, Artificial
  chimeric sequence (rat/human)
QLQLKESGPGLVQPSQTLSLTCTVSELSLTSNSITWIRQPPGRGLEWMGMIWSNGDTDYNSAFTSRLSISRDTSKSQVFLKMNSLQTEDSAMYFCARYNYYFDYWGQGVMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 217 aa, Artificial
  chimeric sequence (rat/human)
DIVLTQAPVLAVSLEQRATISCKASQNVDNRGVSYVHWYQQKPRQQPKLLIYKGSNLAFGVPARFSGSGSRTDFTLTIDPVETDDFATYYCQQSKGHPDTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 115 aa, Rattus norvegicus
QLQLKESGPGLVQPSQTLSLTCTVSELSLTSNSITWIRQPPGRGLEWMGMIWSNGDTDYNSAFTSRLSISRDTSKSQVFLKMNSLQTEDSAMYFCARYNYYFDYWGQGVMVTVSS

SEQ ID NO: 7 - protein, 115 aa, Artificial
  Synthesized sequence of HURDIVHv1
QLQLQESGPGLVKPSQTLSLTCTVSGLSLTSNSITWVRQAPGQGLEWMGMIWSNGDTDYNSAFTSRFTISRDNSKNTLYLQMNSLKTEDTAVYYCARYNYYFDYWGQGTLVTVSS

SEQ ID NO: 8 - protein, 115 aa, Artificial
  Synthesized sequence of HURDIVHv2
QLQLQESGPGLVKPSQTLSLTCTVSGLSLTSNSITWVRQAPGQGLEWMGMIWSNGDTDYSTSLKSRLTISKDTSKSQVVLTMTNMDPVDTATYYCARYNYYFDYWGQGTLVTVSS

SEQ ID NO: 9 - protein, 115 aa, Artificial
  Synthesized sequence of HURDIVHv3
QLQLQESGPGLVKPSQTLSLTCTVSGLSLTSNSITWVRQAPGQGLEWMGMIWSNGDTDYNPSLKSRVTISVDTSKNQFSLKLSSVTAADTAVYFCARYNYYFDYWGQGTLVTVSS

SEQ ID NO: 10 - protein, 115 aa, Artificial
  Synthesized sequence of HURDIVHv4
QLQLQESGPGLVKPSQTLSLTCTVSGLSLTSNSITWIRQPPGKGPEWMGMIWSNGDTDYNSAFTSRFTISRDNSKNTLYLQMNSLKTEDTAVYYCARYNYYFDYWGQGTLVTVSS

SEQ ID NO: 11 - protein, 115 aa, Artificial
  Synthesized sequence of HURDIVHv5
QLQLQESGPGLVKPSQTLSLTCTVSGLSLTSNSITWIRQPPGKGPEWMGMIWSNGDTDYSTSLKSRLTISKDTSKSQVVLTMTNMDPVDTATYYCARYNYYFDYWGQGTLVTVSS

SEQ ID NO: 12 - protein, 115 aa, Artificial
  Synthesized sequence of HURDIVHv6
QLQLQESGPGLVKPSQTLSLTCTVSGLSLTSNSITWIRQPPGKGPEWMGMIWSNGDTDYNPSLKSRVTISVDTSKNQFSLKLSSVTAADTAVYFCARYNYYFDYWGQGTLVTVSS

SEQ ID NO: 13 - protein, 110 aa, Rattus norvegicus
DIVLTQAPVLAVSLEQRATISCKASQNVDNRGVSYVHWYQQKPRQQPKLLIYKGSNLAFGVPARFSGSGSRTDFTLTIDPVETDDFATYYCQQSKGHPDTFGAGTKLELK

SEQ ID NO: 14 - protein, 111 aa, Artificial
  Synthesized sequence of HURDIVKv4
DIVLTQSPATLSLSPGERATLSCKASQNVDNRGVSYVHWYQQKPGQAPKLLIYKGSNLAFGVPARFSGSGSGTDFTLTISSLQPEDFATYYCQQSKGHPDTFGAGTKLELK

SEQ ID NO: 15 - protein, 111 aa, Artificial
  Synthesized sequence of HURDIVKv5
DIVLTQSPATLSLSPGERATISCKASQNVDNRGVSYVHWYQQKPGQAPKLLIYKGSNLAFGVPARFSGSGSGTDFTLTISSLQPEDFATYYCQQSKGHPDTFGAGTKLELK

SEQ ID NO: 16 - protein, 111 aa, Artificial
  Synthesized sequence of HURDIVKv7
DIVLTQSPATLSLSPGERATLSCKASQNVDNRGVSYVHWYQQKPGQAPKLLIYKGSNLAFGVPARFSGSGSGTDFTLTISSLQPEDFATYFCQQSKGHPDTFGAGTKLELK

SEQ ID NO: 17 - protein, 111 aa, Artificial
  Synthesized sequence of HURDIVKv8
DIVLTQSPATLSLSPGERATLSCKASQNVDNRGVSYVHWYQQKPGQAPKLLIYKGSNLAFGVPSRFSGSGSGTDFTLTISSLQPEDFATYFCQQSKGHPDTFGAGTKLELK

SEQ ID NO: 18 - protein, 111 aa, Artificial
  Synthesized sequence of HURDIVKv9
DIVLTQSPATLSLSPGERATLSCKASQNVDNRGVSYVHWYQQKPGQAPKLLIYKGSNLAFGVPSRFSGSGSGTDFTLTISSLQPEDFATYYCQQSKGHPDTFGAGTKLELK

SEQ ID NO: 19 - protein, 111 aa, Artificial
  Synthesized sequence of HURDIVKv10
DIVLTQSPATLSLSPGERATLSCKASQNVDNRGVSYVHWYQQKPGQAPKSLIYKGSNLAFGVPSRFSGSGSGTDFTLTISSLQPEDFATYFCQQSKGHPDTFGAGTKLELK

SEQ ID NO: 20 - protein, 115 aa, Artificial
  Synthesized sequence of HUR HuVH v1
QVQLQESGPGLVKPSETLSLTCTVSELSLTSNSITWIRQPPGKGLEWIGMIWSNGDTDYNSAFTSRVTISVDTSKNQFSLKLSSVTAADTAVYYCARYNYYFDYWGQGTLVTVSS

SEQ ID NO: 21 - protein, 115 aa, Artificial
  Synthesized sequence of HUR HuVH v2
QVQLQESGPGLVKPSETLSLTCTVSELSLTSNSITWIRQPPGKGLEWIGMIWSNGDTDYNSAFTSRVTISRDTSKNQFSLKLSSVTAADTAVYYCARYNYYFDYWGQGTLVTVSS

SEQ ID NO: 22 - protein, 115 aa, Artificial
  Synthesized sequence of HUR HuVH v3
QLQLQESGPGLVKPSETLSLTCTVSELSLTSNSITWIRQPPGKGLEWMGMIWSNGDTDYNSAFTSRVTISRDTSKNQFSLKLSSVTAADTAVYYCARYNYYFDYWGQGTLVTVSS

SEQ ID NO: 23 - protein, 119 aa, Artificial
  GenBank Accession Number AAB67785-1
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARAEAAAPYYFDYWGQGTLVTVSS

SEQ ID NO: 24 - protein, 111 aa, Artificial
  Synthesized sequence of HUR HuVK v1
EIVLTQSPATLSLSPGERATLSCKASQNVDNRGVSYVHWYQQKPGQAPRLLIYKGSNLAFGIPARFSGSGSGTDFTLTISSLEPEDFAVYYCQQSKGHPDTFGQGTKVEIK

SEQ ID NO: 25 - protein, 111 aa, Artificial
  Synthesized sequence of HUR HuVK v2
EIVLTQSPATLSLSPGERATLSCKASQNVDNRGVSYVHWYQQKPGQAPRLLIYKGSNLAFGVPARFSGSGSRTDFTLTISSLEPEDFATYYCQQSKGHPDTFGQGTKVEIK

SEQ ID NO: 26 - protein, 111 aa, Artificial
  Synthesized sequence of HUR HuVK v3
EIVLTQSPATLSLSPGERATISCKASQNVDNRGVSYVHWYQQKPGQQPKLLIYKGSNLAFGVPARFSGSGSRTDFTLTISSLEPEDFATYYCQQSKGHPDTFGQGTKVEIK

SEQ ID NO: 27 - protein, 107 aa, Artificial
  GenBank Accession Number CAD43025
EIVLTQSPATLSLSPGERATLSCRASQSVSSYVAWYQQKPGQAPRLLIYDASNRATGIPARFSGSGSGTDFTLTISSLEPEDFAVYYCQQSNNWPQTFGQGTKVEIK

SEQ ID NO: 28 - protein, 313 aa, Homo sapiens
DKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTN

SEQ ID NO: 29 - protein, 107 aa, Homo sapiens [sequence omitted]

SEQ ID NO: 30 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 330 aa, Homo sapiens [sequence omitted]

SEQ ID NO: 32 - protein, 442 aa, Artificial
  Synthesized sequence of heavy chain of DEI 5/8
QLQLQESGPGLVKPSQTLSLTCTVSGLSLTSNSITWIRQPPGKGPEWMGMIWSNGDTDYSTSLKSRLTISKDTSKSQVVLTMTNMDPVDTATYYCARYNYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 218 aa, Artificial
  Synthesized sequence of light chain of DEI 5/8
DIVLTQSPATLSLSPGERATLSCKASQNVDNRGVSYVHWYQQKPGQAPKLLIYKGSNLAFGVPSRFSGSGSGTDFTLTISSLQPEDFATYFCQQSKGHPDTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC